(19) United States
(12) Patent Application Publication
(10) Pub. No.: US 2016/0235219 A1
(43) Pub. Date: Aug. 18, 2016

(54) PRODUCT DISPLAY SYSTEM PROVIDING PRODUCT HUMIDIFICATION

(71) Applicants: James Alan AAMODT, Hood River, OR (US); John William MAYER, Vancouver, WA (US)

(72) Inventors: James Alan Aamodt, Hood River, OR (US); John William Mayer, Vancouver, WA (US)

(21) Appl. No.: 15/031,185

(22) PCT Filed: Oct. 22, 2014
(86) PCT No.: PCT/US14/61770

§ 371 Date: Apr. 21, 2016

Related U.S. Application Data
(60) Provisional application No. 61/894,287, filed on Oct. 22, 2013.

(51) Int. Cl. A47F 3/04; A23B 7/158; B05B 7/24; A23B 4/30

(52) U.S. Cl. CPC A47F 3/0495; A23B 4/30; A23B 7/158; B05B 7/2491

(57) ABSTRACT

A fogger display unit can include a first deflector having an outer surface and an inner surface. The first deflector can have any shape forming a cavity, for example a hemispherical dome shape. The first deflector is positioned above a display case that includes one or more display decks on which consumer products are displayed. A fogger generates a fog that is exhausted towards the inner surface of the first deflector. The first deflector deflects the fog flow downward across the consumer products on the display deck. The fogger display unit can include a second deflector having an outer surface and an inner surface. The inner surface of the second deflector is proximate the outer surface of the first deflector forming a channel therebetween. Air, forced through the channel by an air handler, flows downward as an air curtain about the periphery of the display case.

### BACKGROUND

1. Technical Field

The present disclosure generally relates to the field of product displays.

2. Description of the Related Art

Display cases for displaying items take a large variety of forms. Display cases will typically include one or more decks (e.g. shelves) on which items for display may be arranged. Display cases may include opaque or transparent materials. Display cases typically have at least one open portion which provides access to an interior from an exterior of the display case. For example, a display case may be open along a front of the display case, a top of the display case, and/or along sides or a rear of the display case. In self-serve retail environments, the open portion allows consumers to access and retrieve the displayed items from the display case.

In many retail environments, perishable items are displayed in the display cases. For example, perishable items such as produce including various fruits and vegetables are typically displayed using consumer-accessible display cases in which the produce is arranged. These display cases may or may not include a refrigeration unit to cool or chill the produce as a way of preserving and/or extending the shelf life of the produce.

In some instances, produce displays also include an irrigation system such as one or more sprayer systems that intermittently or periodically spray water on the surface of the produce. Due to the volume of water discharged during such irrigation periods such systems also may include a consumer “warning” system (e.g., recorded peals of thunder and flashing strobe lights) or similar to alert consumers to the imminent water discharge. While spray irrigation systems assist in maintaining the freshness of some types of produce, such systems are limited in scope, maintenance intensive, consume considerable quantities of water, provide water droplets and residue conducive to microbial growth, including spoilage organisms such as pseudomonas, on produce and surfaces in the display case, and result in unintended soakings of unwary consumers.

Retail display cases for perishable items such as produce, fish, poultry and meats typically include a display deck positioned for easy viewing by consumers. In some instances, some display cases, typically meats, fish, and poultry are partially or completely enclosed by transparent panels that allow the consumer to view, but not select the offered products. In such instances, store personnel will access the interior space of the display case and retrieve products at the consumer's request.

In other instances, display cases such a produce display cases, are open to the consumer, allowing the consumer to select one or more desired products. Such produce display cases can take many forms depending on the décor and/or theme of retail establishment. Typically produce cases are either a freestanding “island” style in which consumers can access the produce from more than one side of the display case or a “bookcase” style, usually arranged along a wall, in which consumers can access the produce from only one side (e.g., front).

While consumers prefer the ability to select their own products, the generally open environment in which produce is typically displayed is problematic for several reasons. First, produce is perishable, with some produce sensitive to heat, some sensitive to dryness, and yet others sensitive to both heat and dryness. Consumers are selective and produce that is showing signs of distress such as wilting, discoloration, or physical degradation due to heat and/or dryness will generally remain unsold and represent a cost and/or lost revenue to the retailer. To counteract the effects of heat, some produce display cases are equipped with refrigeration equipment to maintain a cooler than ambient case temperature in the environment about the produce. The effectiveness of such refrigeration equipment is adversely impacted by the open nature of many produce display cases.

To counteract the effects of dryness, some produce display cases are equipped with water spray irrigation systems that periodically spray water on the produce in the display case. While helpful in alleviating some of the effects of dryness, such water sprays consume a considerable quantity of water, provide water droplets and residue conducive to microbial growth, including spoilage organisms such as pseudomonas, on produce and surfaces in the display case, and require frequent maintenance such as adjustment of spray heads which become clogged or are accidentally repositioned by consumers. Such spray irrigation systems may be viewed as an inconvenience by consumers who have to wait until the end of a spray cycle to safely access the displayed produce. Additionally, while current display cases are able to mitigate to an extent the deleterious effects of heat and dryness on produce, such systems are unable to address issues such as contamination (e.g., Salmonella, E. coli, and listeria) that may be present on some produce (e.g., bean sprouts).

### BRIEF SUMMARY

Produce benefitting from a humid display environment may be stocked on display decks in a fogger display unit. A fogger display unit includes a first deflector positioned above the display deck and a fogger subsystem. The first deflector can have any shape or configuration that includes an upper surface and an opposed lower surface, and which angles outwardly in a downward direction. The first deflector can be fabricated as a continuous or segmented curved structure that provides a cavity within the deflector. A hemispherical dome positioned above an island type produce display case is an example first deflector. A polygonal, multi-faceted, awning positioned along all or a portion of a length of a bookcase type produce display case is also an example first deflector. The first deflector may be transparent, semi-transparent, or opaque.

A liquid, which can include water, either from a reservoir or from a clean water source (e.g., a municipal potable water system) is optionally passed through one or more filters and one or more sterilization systems (i.e., ultraviolet light purifier) prior to introduction to the fogger. The fogger nebulizes the feed liquid and passes the nebulized feed liquid via a conduit to a point proximate the first deflector. The nebulized liquid tends to follow the inside surface of the first deflector, resulting in a generally downward flow of nebulized liquid across the produce arranged on the display deck of the display case. The nebulized liquid can include a condensing liquid (i.e., a “wet” fog) or a non-condensing liquid (i.e., a “dry” fog). Where condensing liquids provide at least a portion of the nebulized liquid, one or more drains may be positioned below the display deck to capture condensate and route the condensate to an appropriate discharge point such as a sanitary sewer. A nebulized liquid fog provides a micron droplet size, droplet density and droplet volume that advantageously creates an environment where the produce stomata open, thereby allowing the plants to rehydrate. Additionally, the presence of the nebulized liquid fog provides an evaporative cooling effect, thereby reducing refrigeration demands and the resultant energy costs associated therewith.

In some instances, the liquid may include one or more liquids having one or more desirable biocidal properties. Such liquids may advantageously provide a measure of protection against historical produce-borne pathogens such as Salmonella, E. coli, and listeria. Such liquids may also advantageously provide a measure of protection against human-borne pathogens, such as cold and flu viruses, that may be inadvertently introduced when consumers handle or otherwise interact with the produce. The presence of such liquids having biocidal properties in a nebulized form is particularly advantageous as such a form permits the liquid to flow not just across the surface of the produce, but also to penetrate into the cracks, crevices, and gaps in, around, and between the displayed produce.

In addition to the above described features, one or more second deflectors may be used on some or all produce display cases to provide an air curtain that advantageously assists in retaining the nebulized liquid or fog proximate the produce in the display case. In some instances, the air providing some or all of such an air curtain may be refrigerated or similarly cooled to assist in maintaining an lower than ambient temperature within the display case. A portion of the inner surface of the second deflector is positioned proximate a portion of the outer surface of the first deflector to form a flow channel therebetween. The second deflector can have any shape or configuration that includes an upper surface and an opposed lower surface, which angles outwardly in a downward direction, and which more-or-less follows the shape and/or contour of the first deflector. The second deflector can be fabricated as a continuous or segmented curved structure that provides a cavity within the deflector. A hemispherical dome positioned above a similar hemispherical dome shaper first deflector is an example second deflector. A polygonal, multi-faceted, awning positioned above a similar polygonal, multi-faceted, awning shaped first deflector is also an example second deflector. The second deflector may be transparent, semi-transparent, or opaque. A void, providing a flow channel through which air may pass, exists between the outer surface of the first deflector and inner surface of the second deflector.

The flow channel between the first deflector and the second deflector is fluidly coupled to an air handler that forces air to flow through the channel. The forced air exits the flow channel in a more-or-less vertical downward direction proximate a lower edge of the first deflector, the second deflector or both the first and second deflector. In some instances, the display deck of the display case may have one or more inlets through which at least a portion of the forced air flows for return to the air handler. Such return air beneficially assists in reducing the cooling load placed on an optional cooler or refrigeration unit when cool or chilled forced air provides an air curtain that maintains the display case at a below ambient temperature.

A fogging system for use with a retail display case, the retail display case having at least one deck and a periphery, at least a portion of the deck of the retail display case accessible by consumers along at least a portion of the periphery may be summarized as including: a first deflector positionable over at least the deck of the retail display case, the first deflector having an upper surface and a lower surface, the lower surface which angles outwardly from a top of the first deflector to a bottom of the first deflector to form a cavity; a fogger subsystem, the fogger subsystem having at least one outlet, the at least one outlet positioned to dispense a fog toward the lower surface of the first deflector; and at least one reservoir, the at least one reservoir communicatively coupled to provide at least one of water or at least one biocide agent to the fogger subsystem.

The fogging system may further include: a second deflector positionable over at least the deck of the retail display case, the second deflector having a lower surface, the lower surface positioned with respect the upper surface of the first deflector to form at least one channel therebetween, the channel open to an ambient environment at least proximate a lower edge of at least one of the first or the second deflector; and an air mover subsystem fluidly communicatively coupled to drive air through the at least one channel to form an air curtain. The at least one channel may extend about the at least a portion of the periphery of the retail display case which is accessible by consumers, and the air curtain may encompass the at least a portion of the periphery of the retail display case which is accessible by consumers. The at least one fogger system may include a fogging subsystem fan. The first deflector may be nested in the second deflector. The first deflector and the second deflectors may be transparent. The fogging system may further include: at least one light source positioned carried by at least one of the first or the second deflectors. The fogging system may further include: at least one light source oriented to illuminate at least one of the first or the second deflectors. The first deflector may be a dome and the lower surface of the first deflector may be concave, and the second deflector may be a dome and the lower surface of the second deflector may be concave. The second deflector may have a perimeter, and the perimeter of the second deflector may be coextensive with a perimeter of the deck of the retail display case. The fogging system may further include: a water reservoir fluidly communicatively coupled to provide water from the water reservoir to the fogger subsystem. The fogging system may further include: a biocide agent reservoir communicatively coupled to provide a biocide agent from the biocide agent reservoir to the fogger subsystem. The fogging system may further include: a biocide agent reservoir communicatively coupled to provide a biocide agent from the biocide agent reservoir to the fogger subsystem. The fogging system may further include: a biocide agent reservoir communicatively coupled to provide a biocide agent in a dry powder form from the biocide agent reservoir to the fogger subsystem, the dry fogger system including at least one ultrasonic nebulizer operable to selectively disperse the biocide agent as a dry fog. The at least one fogger system may include an air mover. The first deflector may be transparent. The first deflector may be a dome and the lower surface of the first deflector may be concave. The fogging system may further include: at least one moisture sensor positioned to detect moisture within a volume extending upwardly from the at least one deck of the retail display case and below the first deflector. The fogging system may further include: a fog density sensor to determine the fog volume proximate the at least one deck of the retail display case and below the first deflector. The fogging system may further include: at least one biocide agent sensor positioned to detect a volume or a density of biocide agent within a volume extending upwardly from the at least one deck of the retail display case and below the first deflector.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a cross-sectional elevation of an island-type fogger display system that includes a fogger unit to nebulize a liquid, according to one illustrated embodiment.

FIG. 2 is a cross-sectional elevation of an island type fogger display system that includes an air curtain and a fogger unit to nebulize a liquid, according to one illustrated embodiment.

FIG. 3 is a cross-sectional elevation of a bookcase-type fogger display system that includes a fogger unit to nebulize a liquid, according to one illustrated embodiment.

FIG. 4 is a cross-sectional elevation of a bookcase-type fogger display system that includes an air curtain and a fogger unit to nebulize a liquid, according to one illustrated embodiment.

FIG. 5 is a cross-sectional elevation of a pendant-type fogger display system that includes a fogger unit to nebulize a liquid, according to one illustrated embodiment.

FIG. 6 is a cross-sectional elevation of a pendant-type fogger display system that includes an air curtain and a fogger unit to nebulize a liquid, according to one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with specific construction details of available industrial or commercial equipment such as product display cases, foggers, nebulizers, air handlers, commercial refrigeration units, humidifiers, dehumidifiers, and air conditioners have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless the context makes clear otherwise, the terms biocide agent refers to a biocide whether dry, in solution, or in gaseous form. Thus, nebulizing or nebulization of biocide agent may, for example include nebulizing or nebulization of a dry form of the biocide agent to produce fine particles thereof, or may include nebulizing or nebulization of a fluid form of a biocide agent, for example a biocide agent solution in which a quantity of a biocide agent is mixed in a quantity of fluid (e.g., water).

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

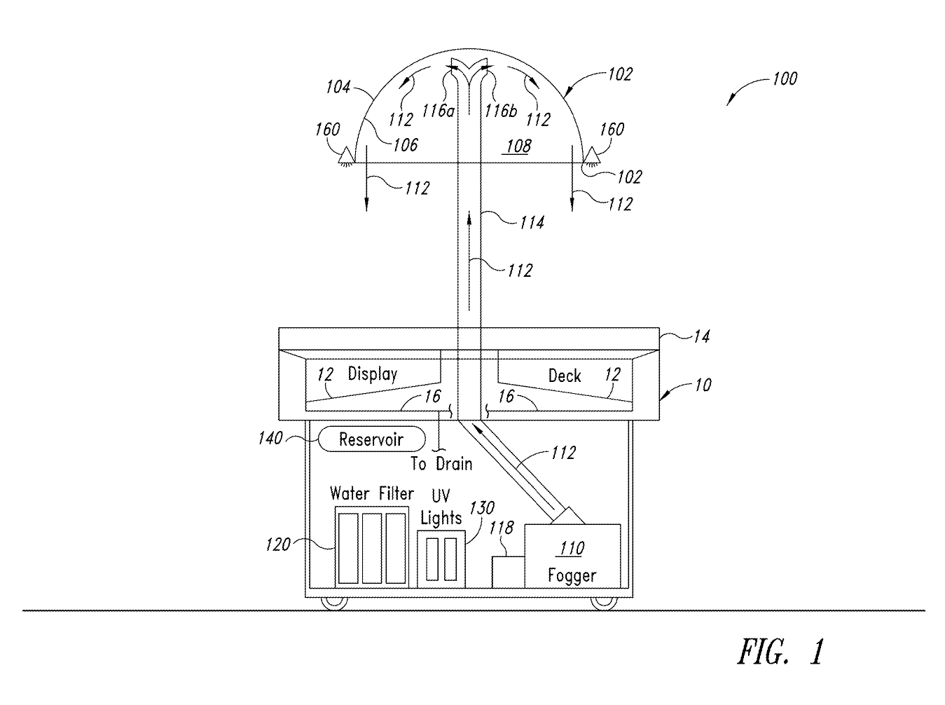

FIG. 1 shows an island-type fogger display system 100 according to one illustrated embodiment. The system 100 includes a first deflector 102 positioned above an island-type display case 10. The island-type display case 10 includes one or more display decks 12 each having a defined periphery 14 that may or may not include a vertical wall or lip to retain produce or other products placed on the respective display deck 12. The one or more display decks 12 may be inclined, sloped, and/or contoured to direct all or a portion of the condensate, water, or other fluids that may accumulate on the surface of the display deck 12 to one or more drains 16.

The first deflector 102 includes an upper surface 104 and a lower surface 106 that angle outwardly from a top of the first deflector 102 to the bottom of the first deflector 102. In one instance, the first deflector 102 may be in the form of a hemispherical dome as depicted in FIG. 1. In other instances, the first deflector 102 may be in the form of a partial polyhedron having any number (e.g., two or more) regular or irregular planar or curved faces. The concave inner surface 106 of the first deflector 102 forms a cavity 108.

A fogger 110 nebulizes, vaporizes, or otherwise atomizes one or more liquids to produce a fog 112 that is directed via one or more fluid conduits 114 to one or more fogger outlets 116a-116b (collectively, “outlets 116”) positioned to dispense or otherwise exhaust the fog 112 towards the lower surface 106 of the first deflector 102. As used herein, the terms “fog” and “nebulize” means the atomization or breaking up of a fluid into sufficiently fine particles so as to produce a fine spray or mist. In some instances, prior to entering the fogger 110, the fluid used to produce the fog 112 may pass through one or more filtration devices 120 and/or one or more sterilizers 130. In some instances, the fogger 110 nebulizes a liquid that includes or consists entirely of water. In other instances, the fogger 110 nebulizes a fluid that includes one or more biocidal agents useful for disinfecting and/or preserving the products displayed on the display decks 12 of the display case 10.

In some instances, the fogger 110 nebulizes one or more condensing fluids, such as water, to produce a “wet fog” that may, depending on environmental conditions, tend to condense on surfaces. In other instances, the fogger nebulizes one or more non-condensing fluids to produce a “dry-fog” that does not tend to condense on surfaces. Since the fog 112 is heavier than air, upon exiting the first deflector 102, the fog 112 tends to sink in a downward direction and flow over, across, around, and between the products placed on the display deck 12. Any condensation occurring on the products or on the display deck 12 collects in the display case drain 16.

The first deflector 102 can have any shape that is capable of directing the fog 112 dispensed by the fogger 110 via the conduits 114 in a downward direction or in a direction towards one or more nearby displayed consumer products. Although depicted as a hemispherical dome structure in FIG. 1, any number of other symmetric or asymmetric geometric designs may be used with equal effectiveness. For example, in some instances, a multi-sided pyramidal first deflector 102 structure may be used to better align with the perimeter of triangular, square, or rectangular shaped island-type display cases 10. In other instances, a prismatic first deflector 102 structure (e.g., a pool table lamp type structure) may be used to better align with the perimeter of rectangular or oval shaped island-type display cases 10.

The first deflector 102 can include any material, including one or more transparent, translucent, or opaque materials or any combination thereof. Such materials may include fabrics, flexible polymeric materials, rigid polymeric materials, or even metals. The type, color, and optical properties of the first deflector 102 material may be selected at least in part to integrate the appearance of the display case 10 into an overall décor, theme, or color scheme. In some instances, the first deflector 102 may include one or more passive flow guidance or passive turbulence producing devices such as strakes, vanes, creases, blades, or similar disposed in or on all or a portion of the inner surface 106.

The fogger 110 can include one or more devices, systems, or combination of systems and devices capable of receiving one or more liquids and nebulizing or atomizing at least a portion of the received one or more liquids to provide the fog 112. In some instances, the liquid can include water in whole or in part. In some instances, the liquid can include one or more biocidal agents useful for sterilizing and/or preserving the products in the display case 10. In at least some implementations, the fogger 110 can include one or more “cool mist” or similar (e.g., vibrating membrane) type devices able to produce a quantity of fog 112 without requiring significant, or even any, heating of the fluid. In some instances, the fogger 110 can include one or more air movers or similar devices such as a fan to force exhaust the fog 112 via the one or more outlets 116.

The fogger 110 includes one or more transducers selectively operable to nebulize or fog the fluid provided to the fogger 110. In practice the fogger 110 produces a fine mist or fog of water droplets that may or may not carry one or more additional substances such as a biocide or a preservative.

The transducer(s) in the fogger 110 may take a variety of forms, including one or more piezoelectric transducers, vibration table associated with one or more motors and/or one or more transmissions, oscillating or vibrating meshes/membranes, oscillating or vibrating magnets, jets and source of a compressed fluid to drive the jet, to name a few. Piezoelectric transducers typically are driven by a high frequency voltage source, and may mechanically impart its oscillatory or vibratory motion directly to the fluid. Jet nebulizers, sometimes referred to as atomizers, typically employ a jet port which emits a fluid (e.g., air or oxygen) at a high velocity, and possibly with significant turbulence, into the fluid to be delivered as a fog. The fluid is supplied to the jet port at an elevated pressure relative to the ambient pressure, typically via conduit, valves and a compressor.

The transducer(s) in the fogger 110 may, for example, be used to break up fluid (e.g., water) to produce the fog 112 (i.e., mixture of gas and particle) dispensed toward the inner surface 106 of the first deflector 102. An aerosol may take a variety of forms. For example, an aerosol may take the form of vaporized water entrained in warm air, to produce the fog 112. The transducer(s) in the fogger 110 may vibrate or oscillate or produce vibration or oscillation at one or more desired or defined frequencies. For example the transducer(s) in the fogger 110 may vibrate at one or more ultrasonic frequencies to obtain sufficiently nebulization to achieve distribution as a dry fog 112.

The fogger 110 dispenses fog 112 towards the inner surface 106 of the first deflector 102 at one or more points. At times, a single conduit 114 may dispense or otherwise discharge the fog 112 towards a single point on the inner surface 106 of the first deflector 102. At other times, more than one conduit 114 may be used to dispense or otherwise discharge the fog 112 towards multiple points on the inner surface 106 of the first deflector 102.

In at least some instances, the fluid supplied to the fogger 110 may be provided via a connection to a fluid supply system such as a municipal or utility potable water system. In such instances, the fluid may pass through one or more pretreatment systems prior to introduction to the fogger 110. For example, the fluid may pass through one or more filters 120 such as one or more spiral wound filament filters to remove dirt and other sediment that may potentially foul or scale out in the fogger 110. In another example, the fluid may pass through one or more filters 120 that incorporate an ion exchange or similar resin to remove calcium, magnesium, and other potentially scale forming ions from the fluid prior to introduction to the fogger 110. In yet another example, the fluid may pass through one or more filters 120 that incorporate one or more reverse osmosis or similar membrane purifiers to remove some or all of the contaminants from the fluid prior to introduction to the fogger 110. In some instances, the filter 120 can include combinations of treatment technologies including some or all of activated carbon, filament filters, ion exchange resins, and membrane filters.

The fogger 110 may include one or more control systems 118. Each control system 118 may include a control device such as a processor or digital signal processor that is communicably coupled to one or more sensors and one or more output devices. Each control system 118 may optionally include one or more local and/or remote output devices to provide information and alerts to operating personnel. The one or more sensors may include one or more humidity sensors, one or more fog density sensors, one or more biocide-specific sensors, one or more temperature sensors, or combinations thereof sufficient to monitor one or more operating parameters of the fogger system 100.

In some instances, the control system 118 may cause the fogger 110 to operate only a portion of the time (e.g., 30 minutes of operation every 60 minutes). In such instances, the control system 118 may receive an input indicative of the fog density and/or relative humidity measured by a sensor proximate the display deck 12 and may cause the fogger 110 to operate when the measured fog density and/or relative humidity falls outside a defined range. In some instances, the control system 118 may reduce the output of the fogger at times when the display is inaccessible to consumers (e.g., during the hours when the store is closed). In some instances, the control system 118 may receive an input indicative of the biocide concentration measured by a biocide-specific sensor proximate the display deck 12 and may cause the fogger 110 to operate when the measured biocide concentration falls outside a defined range. In some instances, the control system 118 may receive data from one or more external sources, for example an external sensor signal carrying data indicative of the ambient relative humidity of the environment surrounding the display case 10, which the controller 118 may use to alter or adjust the operation of the fogger 110.

In some instances, the control system 118 may monitor conditions in the fluid filter 120 to alert operating personnel of the need to replenish, replace, or clean supplies and/or components in the fluid filters 130. In some instances, the control system 118 may monitor conditions in the sterilizer 130 to alert operating personnel of the need to replenish, replace, or clean supplies and/or components in the sterilizer 130. In some instances, the control system 118 may monitor conditions in the reservoir 140 to alert operating personnel of the need to replenish, replace, or clean supplies and/or components in the reservoir 140.

In some instances, the control system 118 may include one or more proximity sensing devices capable of detecting a consumer's approach to the display case 10. In such instances, while the consumer is proximate the display case 10 the control system 118 may limit or even halt the operation of the fogger 110 to provide the consumer with access to the products on the display deck 12 unhindered by the fog 112.

The one or more filters 120 may be accessible from the exterior of the display case 10 to facilitate inspection, cleaning, and/or replacement. In at least some implementations one or more indicators providing information indicative of the condition and/or performance of the one or more filters 120 may be visible from the exterior of the display case 10.

In some instances, the fluid may pass through one or more sterilizers 130 prior to introduction to the fogger 110. In one example, the fluid may pass through one or more sterilizers 130 that include one or more ultraviolet (“UV”) light generators to render harmless all or a portion of any microbiological growth in the fluid prior to introducing the fluid to the fogger 110. In another example, the fluid may pass through one or more sterilizers 130 that include one or more ozonizers to render harmless all or a portion of any microbiological growth present in the fluid prior to introducing the fluid to the fogger 110.

The one or more sterilizers 130 may be accessible from the exterior of the display case 10 to facilitate inspection, cleaning, and/or replacement. In at least some implementations one or more indicators providing information indicative or representative of the condition and/or performance of the one or more sterilizers 130 may be visible from the exterior of the display case 10.

In some instances, one or more reservoirs 140 may be fluidly coupled to the one or more foggers 110. The reservoirs 140 may be designed to hold water, and optionally to hold one or more biocide or preservation solutions. For example, the system 100 may include two reservoirs, a first 140a to hold a quantity of water and a second 140b to hold a quantity of biocide solution that is mixed with the water prior to introduction to the fogger 110. In other implementations, the system 110 may include a water reservoir 140 to hold water to mix with a dry (e.g., powdered) biocide agent to create a biocide solution prior to introduction to the fogger 110.

The interior of the one or more reservoirs 140 may be accessible from the exterior of the display case 10 to facilitate cleaning and/or replenishment of the reservoir 140. In at least some implementations one or more indicators providing information indicative of the condition of and/or level in the one or more reservoirs 140 may be visible from the exterior of the display case 10. Some or all of the reservoirs 140 may be equipped with one or more metering devices to meter the contents of the reservoir into the fluid supplied to the fogger 110 or to meter the contents of the reservoir directly to the fogger 110. The metering devices may take a variety of forms including one or more unpowered forms such as an eductor or venturi or one or more powered forms such as a piston pump, a plunger pump, or a peristaltic pump. Other feed devices may also be used either alone or in combination.

One or more lighting units 160 may be physically coupled to the first deflector 102. The lighting units 160 may include line or reduced voltage lighting devices intended to illuminate the products placed on the display deck 12 of the display case 10. The lighting units 160 may include line or reduced voltage decorative or accent lights, such as various or single color light emitting diode (LEDs) or similar. In some instances, all or a portion of the lighting units 160 may include one light emitting devices generating light in a spectrum useful for plant photosynthesis. In some instances, other lighting units 160 may be mounted internal to the first deflector 102. For example, a small, low-power, laser projector may be mounted internal to the first deflector 102 to take advantage of the fog 112 beneath the first deflector 102 to project one or more images or messages in the space between the edge 109 of the first deflector 102 and the display deck 12.

In at least some instances, all or a portion of the fogger system 100 may be of a construction that facilitates disassembly and sterilization to comply with local, municipal, state, or federal health requirements related to equipment designed or intended for use in consumer food contact service.

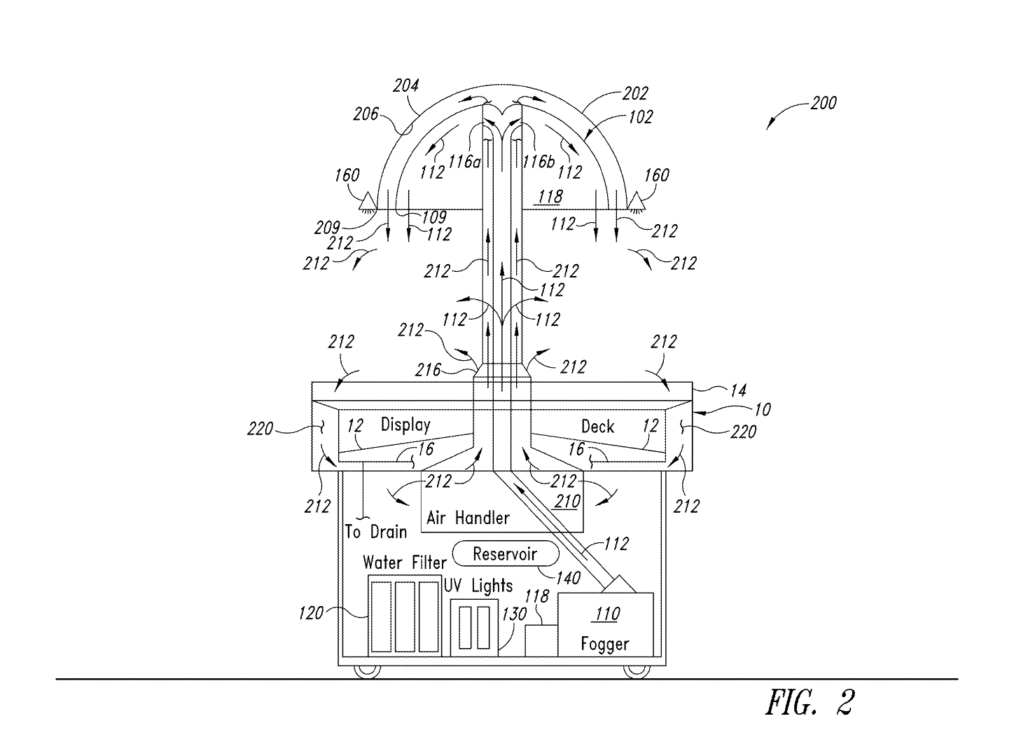

FIG. 2 shows an island-type fogger display system 200 according to one illustrated embodiment. The system 200 includes a second deflector 202 having an outer surface 204 and an inner surface 206. The second deflector 202 is disposed proximate the outer surface 106 of the first deflector 102. The positioning of the second deflector 202 relative to the first deflector 102 forms a channel 208 between at least a portion of the outer surface 104 of the first deflector 102 and at least a portion of the inner surface 206 of the second deflector 202. The channel 208 the channel 208 formed between the first deflector 102 and the second deflector 202 opens to the ambient environment proximate an edge 109 of the first deflector 102, proximate an edge 209 of the second deflector 202, or proximate the edges 109, 209 of both the first deflector 102 and the second deflector 202, respectively.

The system 200 also includes an air handler 210 to move a volume of air 212 through one or more conduits 214 fluidly coupling the air handler 210 to the channel 208. The air 212 provided by the air handler 210 flows partially or completely through the channel 208 and exits the channel 208 flowing in a downward direction toward the display case 10 below. Thus, at least a portion of the air 212 provided by the air handler 210 provides a downward flowing “curtain” of air 212 about all or a portion of the periphery 14 of the display case 10. In some instances, one or more flow velocity increasing or backpressure enhancing devices may be disposed at the exit of all or a portion of the channel 208. Example flow velocity increasing devices include a flat plate having a number of orifices or apertures penetrating therethrough (i.e., an orifice plate). In some instances, at least a portion of the air 212 exhausted by the air handler 210 exits the one or more conduits 214 via one or more ports 216 proximate the display deck 12 of the display case 10. In some instances, one or more light sources (not shown in FIG. 2) such as one or more LED light sources may be disposed in some portions of the channel 208 to provide a display attractive to consumers. In some instances, one or more aroma producing devices may be disposed in some portions of the channel 208 to provide a display capable of producing an aroma attractive to consumers. For example, an aroma producing device capable of producing a strawberry aroma may be provided above a display of strawberries.

In some instances, one or more digital sign panels visible from the exterior of the second deflector 202 may be disposed within a portion of the channel 208. Such digital sign panels may include one or more backlit liquid crystal display (“LCD”) panels with a backlight installed in the channel 208 or in the cavity 118, one or more light emitting diode (“LED”) panels, one or more flexible LCD panels with a backlight installed in the channel 208 or in the cavity 118, one or more organic LED (“OLED”) panels, one or more polymer LED (“PLED”) panels, or any other current or future developed digital signage. Such digital signage may be used to provide information on the products available in the display case (e.g., “Blueberries $2.99/lb”). Such digital signage may be used to present information on the products available in the display case (e.g., how to pick a good cantaloupe). Such digital signage may be used to present information such as recipes using the products available in the display case (e.g., rhubarb recipes).

In at least some instances, the air handler 210 can include one or more optional conditioning units to alter one or more properties of the air 212 prior to exhausting the air to the one or more conduits 214 In one example, the air handler 210 may include one or more dehumidifiers to reduce the relative humidity of the air 212 prior to exhausting the air 212 to the one or more conduits 214. In another example, the air handler 210 may include one or more humidifiers to increase the relative humidity of the air 212 prior to exhausting the air 212 to the one or more conduits 214. In yet another example, the air handler 210 may include one or more air conditioners to reduce the temperature of the air 212 prior to exhausting the air 212 to the one or more conduits 214.

The control system 118 can control one or more operational aspects and/or parameters of the air handler 210. For example, the control system 118 may reduce the flow of air 212 provided by the air handler 210 when consumers are unable to access the display 10 such as during the hours that the store is closed. In another example, the control system 118 can adjust the output of the refrigeration system in the air handler 210 such that the air 212 provided by the air handler 210 is in a defined temperature range or at a defined temperature setpoint. In another example, the control system 118 can adjust the output of the humidifier in the air handler 210 such that the air 212 provided by the air handler 210 is in a defined humidity range or at a defined humidity setpoint.

In some instances, at least a portion of the air 212 provided as an air curtain via the channel 208 and/or at least a portion of the air 212 exhausted by the ports 216 at the display deck 12 may flow though the display deck 212 into a plenum area 220 beneath the display deck 12. For example, at least a portion of the air 212 may flow through perforated sections of the display deck 212. At least a portion of the air 212 flowing into the plenum area 220 beneath the display deck 12 may provide at least a portion of the make-up air flow to the air handler 210. Recovering at least a portion of the air 212 flowing into the plenum area 220 beneath the display deck 12 may beneficially reduce the energy requirements of the air handler 210, particularly when one or more conditioning units are included in the air handler 210.

The second deflector 202 can have any shape that is capable of directing the air 212 provided by the air handler 210 via the one or more conduits 214 in a downward direction proximate the first deflector 102. Although depicted as a hemispherical dome structure in FIG. 2, any number of other symmetric or asymmetric geometric designs may be used with equal effectiveness to provide the second deflector 202. For example, in some instances, a multi-sided pyramidal second deflector 202 structure may be used to better align with the perimeter of triangular, square, or rectangular shaped island-type display cases 10. In other instances, a prismatic second deflector 202 (e.g., a pool table lamp type structure) may be used to better align with the perimeter of rectangular or oval shaped island-type display cases 10.

The second deflector 202 can include any material, including one or more transparent, translucent, or opaque materials or any combination thereof. Such materials may include fabrics, flexible polymeric materials, rigid polymeric materials, or even metals. The type, color, and optical properties of the second deflector 202 material may be selected at least in part to integrate the appearance of the display case 10 into an overall décor, theme, or color scheme. In some instances, the second deflector 202 may include one or more passive flow guidance or passive turbulence producing devices such as strakes, vanes, creases, blades, or similar disposed in or on all or a portion of the inner surface 206 of the second deflector 202.

In at least some instances, all or a portion of the fogger system 200 may be of a construction that facilitates disassembly and sterilization to comply with local, municipal, state, or federal health requirements related to equipment designed or intended for use in consumer food contact service.

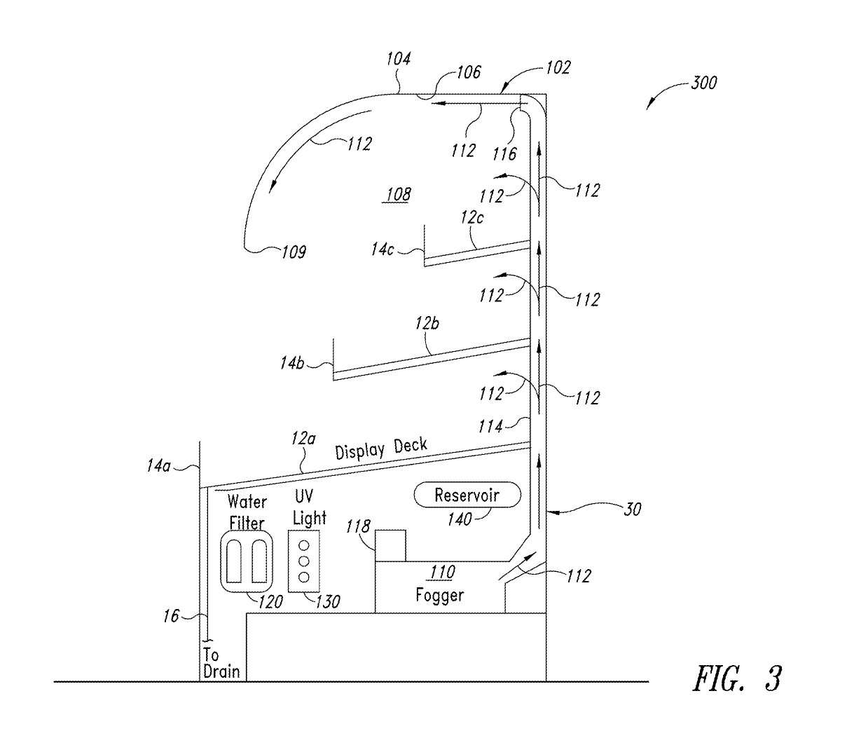

FIG. 3 shows a wall or bookcase-style single or multi-tier display case 30 including an awning-style dome fogger system 300 according to one illustrated embodiment. The wall or bookcase-style single or multi-tier display case 30 includes one or more display decks 12 (three such display decks 12a-12c are depicted in FIG. 3) and has a defined periphery 14 that may or may not include a vertical wall or lip to retain produce or other products placed on the one or more display decks 12. The wall or bookcase-style single or multi-tier display case 30 provides consumer access to products on the one or more display decks 12 from all or a portion of only one side of the display case 30. The system 300 includes a first deflector 102 positioned above a bookcase-style single or multi-tier display case 30. Some or all of the one or more display decks 12 may be inclined to direct condensate, water, or other fluids to one or more drains 16.

The first deflector 102 includes an upper surface 104 and a lower surface 106 that angle outwardly from a top of the first deflector 102 to the bottom of the first deflector 102. In one instance, the first deflector 102 may be in the form of a curved awning as depicted in FIG. 1. In other instances, the first deflector 102 may be in the form of a partial polyhedron having any number (e.g., two or more) regular or irregular planar or curved faces. The concave inner surface 106 of the first deflector 102 form a cavity 108.

The fogger 110 nebulizes, vaporizes, or otherwise atomizes one or more liquids to produce a fog 112 that is directed via one or more fluid conduits 114 to one or more fogger outlets 116 positioned to dispense or otherwise exhaust the fog 112 either proximate or towards the lower surface 106 of the first deflector 102. In some instances, prior to entering the fogger 110, the fluid used to produce the fog 112 may pass through one or more filtration devices 120 and/or one or more sterilizers 130. In some instances, one or more reservoirs 140 each contain one or more liquids for supply to the fogger 110.

In at least some instances, all or a portion of the fogger system 300 may be of a construction that facilitates disassembly and sterilization to comply with local, municipal, state, or federal health requirements related to equipment designed or intended for use in consumer food contact service.

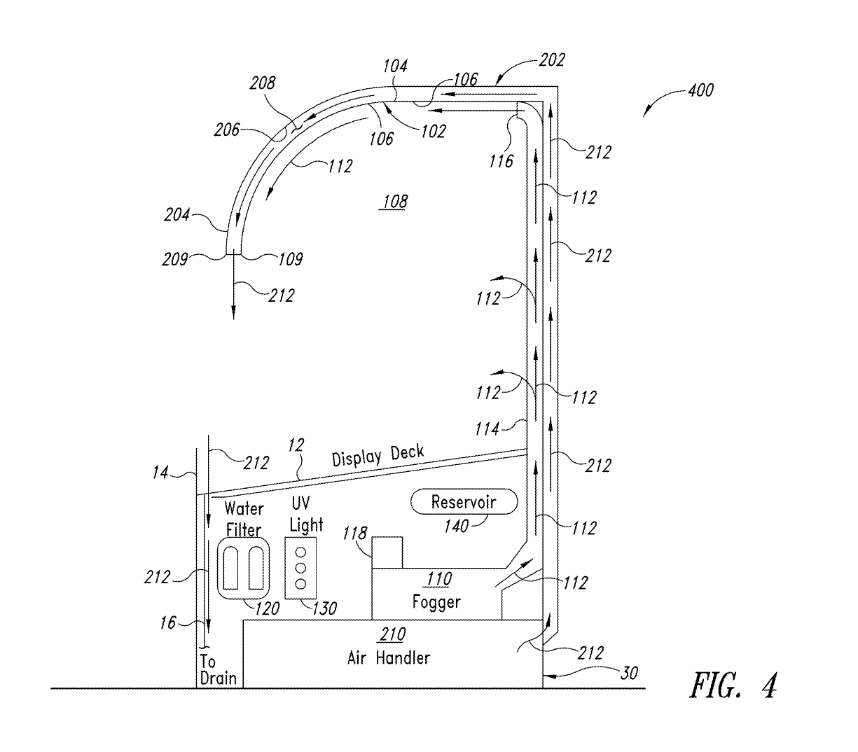

FIG. 4 shows a wall or bookcase-style single or multi-tier display case 30 including an awning-style dome fogger system 400 according to one illustrated embodiment. The wall or bookcase-style single or multi-tier display case 30 includes one or more display decks 12 (three such display decks 12a-12c are depicted in FIG. 3) and has a defined periphery 14 that may or may not include a vertical wall or lip to retain produce or other products placed on the one or more display decks 12. The wall or bookcase-style single or multi-tier display case 30 provides consumer access to products on the one or more display decks 12 from all or a portion of only one side of the display case 30. The system 400 includes a second deflector 202 having an outer surface 204 and an inner surface 206. The second deflector 202 is disposed proximate the outer surface 106 of the first deflector 102. The positioning of the second deflector 202 relative to the first deflector 102 forms a channel 208 between at least a portion of the outer surface 104 of the first deflector 102 and at least a portion of the inner surface 206 of the second deflector 202. The channel 208 formed by the first deflector 102 and the second deflector 202 opens to the ambient environment proximate an edge 109 of the first deflector 102, proximate an edge 209 of the second deflector 202, or proximate the edges 109, 209 of both the first deflector 102 and the second deflector 202, respectively.

The system 400 also includes an air handler 210 to move a volume of air 212 through one or more conduits 214 fluidly coupling the air handler 210 to the channel 208. The air 212 provided by the air handler 210 flows partially or completely through the channel 208 and exits the channel 208 flowing in a downward direction toward the display case 30 below. Thus, at least a portion of the air 212 provided by the air handler 210 provides a downward flowing “curtain” of air 212 about all or a portion of the periphery 14 of the display case 30. In some instances, one or more flow velocity increasing or backpressure enhancing devices may be disposed at the exit of all or a portion of the channel 208. Example flow velocity increasing devices include a flat plate having a number of orifices or apertures penetrating therethrough (i.e., an orifice plate).

In at least some instances, all or a portion of the fogger system 400 may be of a construction that facilitates disassembly and sterilization to comply with local, municipal, state, or federal health requirements related to equipment designed or intended for use in consumer food contact service.

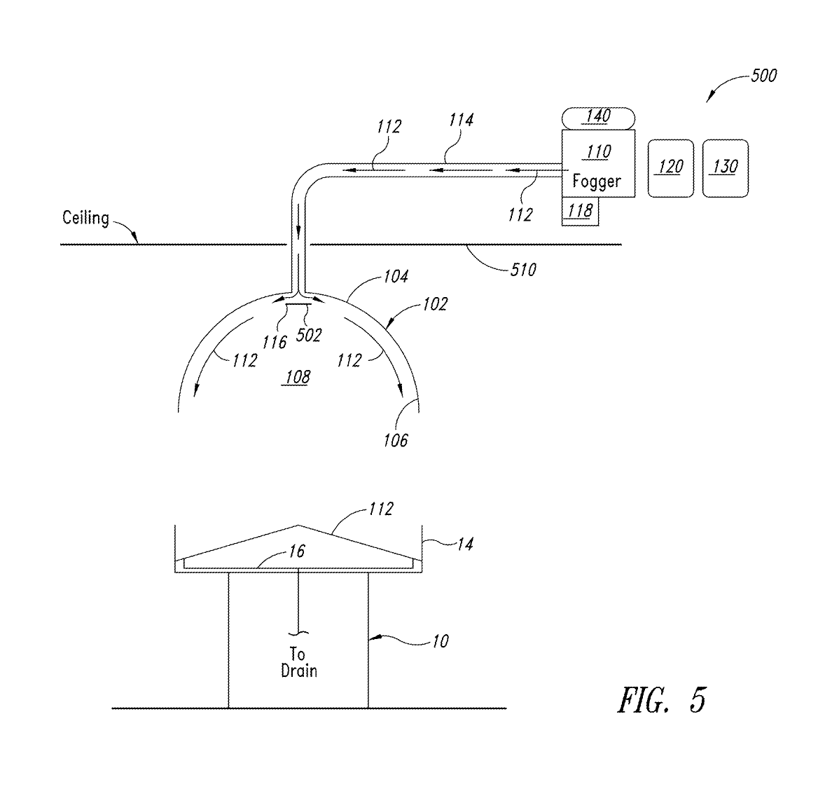

FIG. 5 shows an island-type single or multi-tier display case 10 including a pendant-style fogger system 500 according to one illustrated embodiment. The island-style single or multi-tier display case 10 includes one or more display decks 12 and has a defined periphery 14 that may or may not include a vertical wall or lip to retain produce or other products placed on the one or more display decks 12. The island-style single or multi-tier display case 10 provides consumer access to products on the one or more display decks 12 from all or a portion of the perimeter of the display case 10. The system 500 includes a pendant-style first deflector 102 positioned above the island-style single or multi-tier display case 10. Some or all of the one or more display decks 12 may be inclined to direct condensate, water, or other fluids to one or more drains 16.

The first deflector 102 includes an upper surface 104 and a lower surface 106 that angle outwardly from a top of the first deflector 102 to the bottom edge 109 of the first deflector 102. In one instance, the first deflector 102 may be in the form of a curved awning as depicted in FIG. 5. In other instances, the first deflector 102 may be in the form of a partial polyhedron having any number (e.g., two or more) regular or irregular planar or curved faces. The concave inner surface 106 of the first deflector 102 form a cavity 108.

In some implementations, the first deflector 102 can include one or more impingement device or distributor 502 disposed proximate the outlet 116 of the one or more conduits 114 carrying the fog 112 from the fogger 110. The distributor 502 can direct or otherwise distribute at least a portion of the fog 112 exhausted from the one or more conduits 114 toward the inner surface 106 of the first deflector 102.

In the implementation depicted in FIG. 5, all or a portion of each of the fogger 110, the fluid filters 120, the sterilizers 130, and/or the reservoir 140 may be disposed above the first deflector 502. In some instances, all or a portion of each of the fogger 110, the fluid filters 120, the sterilizers 130, and/or the reservoir 140 may be disposed in a location outside the consumer's view, for example above a ceiling 510.

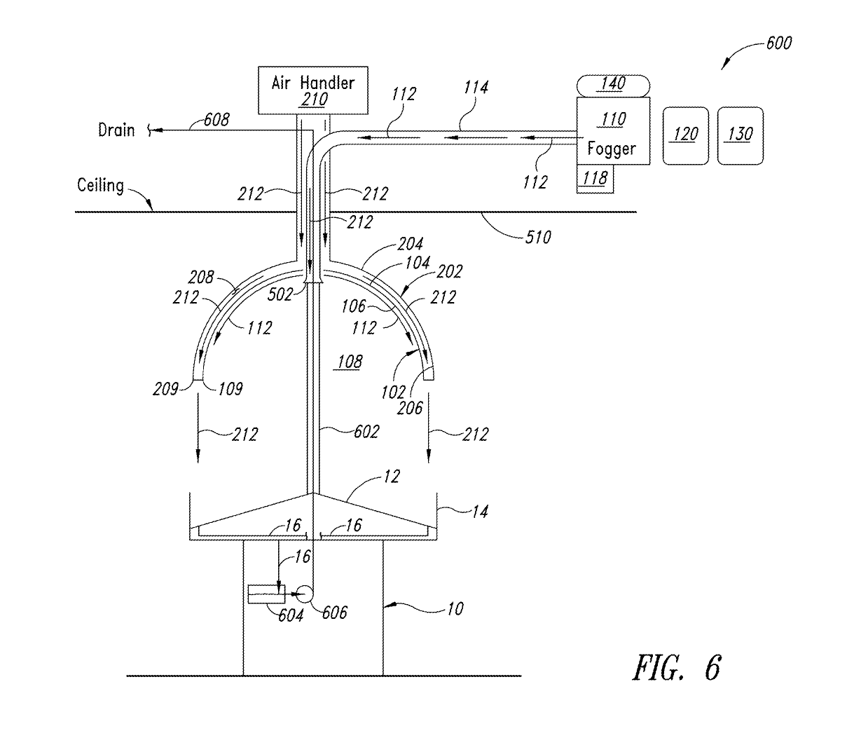

As described in greater detail below with regard to FIG. 6, in some instances, an optional hollow center column (not depicted in FIG. 5) may operably couple the pendant style fogger system 500 with display case 10. In such instances, rather than discharge to a floor drain, water and other liquids collected by the display case drain 16 may be coupled to and discharged through an overhead drain, thereby advantageously eliminating the need for a floor drain connection to the display case 10.

FIG. 6 shows an island-type single or multi-tier display case 10 including a pendant-style fogger system 600 according to one illustrated embodiment. Advantageously, a hollow center column 602 operably couples the display case 10 with a pendant style fogger system 600 such as one or more drains, thereby eliminating the need for below floor drain piping and increasing the flexibility of the system 600. Typically, condensate and other fluids collected on the display decks 12 of a display case 10 require disposal to a sanitary drain via one or more in-floor drainage systems. Such in-floor drainage systems are fixed in place and detrimentally limit placement of the display case 10 to available drain locations rather than locations determined by aesthetics, ergonomics, consumer preference, and/or merchandising. In the pendant-style fogger system 600, liquids collected by the drain 16 are routed to a fluid reservoir 604 located in an out-of-view location such as in the base of the display case 10. A fluid mover such as a pump 606, collocated with the reservoir 604 in the base of the display case 10, periodically transfers the liquid accumulated in reservoir 604 to a remote location via a discharge line 608 routed through the hollow center column 602 coupling the pendant style fogger system 600 to the display case 10. Electricity and any other utilities are also routed from overhead, through the hollow center column 602, to the display case 10. Since the pendant-style fogger system 600 does not require an in-floor drain, such a system advantageously permits the placement of the display case 10 in virtually any location offering adequate overhead space and access to utilities.

The pendant-style fogger system 600 also includes a second deflector 202 having an outer surface 204 and an inner surface 206. The second deflector 202 is disposed proximate the outer surface 106 of the first deflector 102. The positioning of the second deflector 202 relative to the first deflector 102 forms a channel 208 between at least a portion of the outer surface 104 of the first deflector 102 and at least a portion of the inner surface 206 of the second deflector 202. The channel 208 the channel 208 formed between the first deflector 102 and the second deflector 202 opens to the ambient environment proximate an edge 109 of the first deflector 102, proximate an edge 209 of the second deflector 202, or proximate the edges 109, 209 of both the first deflector 102 and the second deflector 202, respectively.

The system 600 also includes an air handler 210 to move a volume of air 212 through one or more conduits 214 fluidly coupling the air handler 210 to the channel 208. The air 212 provided by the air handler 210 flows partially or completely through the channel 208 and exits the channel 208 flowing in a downward direction toward the display case 10 below. Thus, at least a portion of the air 212 provided by the air handler 210 provides a downward flowing “curtain” of air 212 about all or a portion of the periphery 14 of the display case 10. In some instances, one or more flow velocity increasing or backpressure enhancing devices may be disposed at the exit of all or a portion of the channel 208. Example flow velocity increasing devices include a flat plate having a number of orifices or apertures penetrating therethrough (i.e., an orifice plate).

In some instances, one or more light sources (not shown in FIG. 6) such as one or more LED light sources may be disposed in some portions of the channel 208 to provide a display attractive to consumers. In some instances, one or more aroma producing devices may be disposed in some portions of the channel 208 to provide a display capable of producing an aroma attractive to consumers. For example, an aroma producing device capable of producing a strawberry aroma may be provided above a display of strawberries.

In some implementations, the first deflector 102 can include one or more impingement device or distributor 502 disposed proximate the outlet 116 of the one or more conduits 114 carrying the fog 112 from the fogger 110. The distributor 502 can direct or otherwise distribute at least a portion of the fog 112 exhausted from the one or more conduits 114 toward the inner surface 106 of the first deflector 102.

In the system 600 depicted in FIG. 6, all or a portion of each of the fogger 110, the fluid filters 120, the sterilizers 130, the reservoir 140, and/or the air handler 210 may be disposed above the first deflector 502. In some instances, all or a portion of each of the fogger 110, the fluid filters 120, the sterilizers 130, the reservoir 140 and/or the air handler 210 may be disposed in a location outside the consumer's view, for example above a ceiling 510.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other environments, not necessarily the exemplary commercial environment generally described above.

Also for instance, the foregoing detailed description has set forth various embodiments of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one embodiment, the present subject matter may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

In addition, those skilled in the art will appreciate that the mechanisms of taught herein are capable of being distributed as a program product in a variety of forms, and that an illustrative embodiment applies equally regardless of the particular type of physical signal bearing media used to actually carry out the distribution. Examples of physical signal bearing media include, but are not limited to, the following: recordable type media such as floppy disks, hard disk drives, CD ROMs, digital tape, and computer memory.

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet, including but not limited to: U.S. Provisional Patent Application Ser. No. 61/894,287, filed Oct. 22, 2013; U.S. Provisional Patent Application Ser. No. 61/433,774, filed Jan. 18, 2011; Patent Cooperation Treaty Patent Application Ser. No. PCT/US2011/050715, filed Sep. 7, 2011; and U.S. Patent Publication No. US 2013/0183749A1, filed Mar. 7, 2013 and published Jul. 18, 2013, are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fogging system for use with a retail display case, the retail display case having at least one deck and a periphery, at least a portion of the deck of the retail display case accessible by consumers along at least a portion of the periphery, the fogging system comprising:
a first deflector positionable over at least the deck of the retail display case, the first deflector having an upper surface and a lower surface, the lower surface which angles outwardly from a top of the first deflector to a bottom of the first deflector to form a cavity;
a fogger subsystem, the fogger subsystem having at least one outlet, the at least one outlet positioned to dispense a fog toward the lower surface of the first deflector; and
at least one reservoir, the at least one reservoir communicatively coupled to provide at least one of water or at least one biocide agent to the fogger subsystem.

2. The fogging system of claim 1, further comprising:
a second deflector positionable over at least the deck of the retail display case, the second deflector having a lower surface, the lower surface positioned with respect the upper surface of the first deflector to form at least one channel therebetween, the channel open to an ambient environment at least proximate a lower edge of at least one of the first or the second deflector; and
an air mover subsystem fluidly communicatively coupled to drive air through the at least one channel to form an air curtain.

3. The fogging system of claim 2 wherein channel extends about the at least a portion of the periphery of the retail display case which is accessible by consumers, and the air curtain encompasses the at least a portion of the periphery of the retail display case which is accessible by consumers.

4. The fogging system of claim 2 wherein the at least one fogger system includes a fogging subsystem fan.

5. The fogging system of claim 2 wherein the first deflector is nested in the second deflector.

6. The fogging system of claim 2 wherein first deflector and the second deflectors are transparent.

7. The fogging system of claim 2, further comprising:
at least one light source positioned carried by at least one of the first or the second deflectors.

8. The fogging system of claim 2, further comprising:
at least one light source oriented to illuminate at least one of the first or the second deflectors.

9. The fogging system of claim 2 wherein the first deflector is a dome and the lower surface of the first deflector is concave, and the second deflector is a dome and the lower surface of the second deflector is concave.

10. The fogging system of claim 2 wherein the second deflector has a perimeter, and the perimeter of the second deflector is coextensive with a perimeter of the deck of the retail display case.

11. The fogging system of claim 1, further comprising:
a water reservoir fluidly communicatively coupled to provide water from the water reservoir to the fogger subsystem.

12. The fogging system of claim 11, further comprising:
a biocide agent reservoir communicatively coupled to provide a biocide agent from the biocide agent reservoir to the fogger subsystem.

13. The fogging system of claim 1, further comprising:
a biocide agent reservoir communicatively coupled to provide a biocide agent from the biocide agent reservoir to the fogger subsystem.

14. The fogging system of claim 1, further comprising:
a biocide agent reservoir communicatively coupled to provide a biocide agent in a dry powder form from the biocide agent reservoir to the fogger subsystem, the dry fogger system including at least one ultrasonic nebulizer operable to selectively disperse the biocide agent as a dry fog.

15. The fogging system of claim 1 wherein the at least one fogger system includes an air mover.

16. The fogging system of claim 1 wherein first deflector is transparent.

17. The fogging system of claim 1 wherein the first deflector is a dome and the lower surface of the first deflector is concave.

18. The fogging system of claim 1, further comprising:
at least one of either a fog density sensor or a moisture sensor positioned to detect moisture within a volume extending upwardly from the at least one deck of the retail display case and below the first deflector.

19. The fogging system of claim 1, further comprising:
at least one biocide agent sensor positioned to detect a volume or a density of biocide agent within a volume extending upwardly from the at least one deck of the retail display case and below the first deflector.
